(19) United States
(12) Patent Application Publication
(10) Pub. No.: US 2022/0257312 A1
(43) Pub. Date: Aug. 18, 2022

(54) JAW APERTURE POSITION SENSOR FORELECTROSURGICAL FORCEPS

(71) Applicant: Covidien LP, Mansfield, MA (US)

(72) Inventor: David Michael KEFFELER, Niwot, CO (US)

(73) Assignee: Covidien LP, Mansfield, MA (US)

(21) Appl. No.: 17/736,375

(22) Filed: May 4, 2022

Related U.S. Application Data
(63) Continuation of application No. 15/418,809, filed on Jan. 30, 2017.
(60) Provisional application No. 62/288,975, filed on Jan. 29, 2016.

(51) Int. Cl. A61B 18/14; A61B 90/00; A61B 34/35

(52) U.S. Cl. CPC A61B 18/1445; A61B 90/06; A61B 2018/00773; A61B 2090/061; A61B 34/35

(57) ABSTRACT

A surgical instrument including a housing, a shaft, and end effector, and a sensor. The shaft extends distally from the housing. The end effector is disposed at a distal end of the shaft and includes first and second jaw members that are moveable relative between first and second configurations. The first and second jaw members are spaced relative to one another in the first configuration and are closer to one another for approximating tissue in the second configuration. A gap distance is defined between the first and second jaw members. The sensor is positioned within the housing and operable to determine the size of the gap distance. The first and second jaw members configured to be electrical activated to treat tissue between the first and second jaw members when the size of the gap distance is within an acceptable range.

### CROSS REFERENCE TO RELATED APPLICATION

The present application is a continuation of U.S. patent application Ser. No. 15/418,809, filed on Jan. 30, 2017, which claims the benefit of and priority to U.S. Provisional Application Ser. No. 62/288,975, filed on Jan. 29, 2016. The entire disclosures of each of the foregoing applications are incorporated by reference herein.

### BACKGROUND

### 1. Technical Field

The present disclosure relates to electrosurgical forceps and more particularly, to a jaw aperture position sensor for use with an endoscopic or open bipolar and/or monopolar electrosurgical forceps for sealing, cutting, and/or coagulating tissue.

### 2. Discussion of Related Art

Electrosurgical forceps utilize both mechanical clamping action and electrical energy to affect hemostasis by heating the tissue and blood vessels to coagulate, cauterize and/or seal tissue. Electrosurgical forceps may be open forceps for use during open surgical procedures or may be endoscopic forceps for remotely accessing organs through smaller, puncture-like incisions.

Many surgical procedures require cutting or ligating blood vessels or vascular tissue. By utilizing an endoscopic electrosurgical forceps, a surgeon can cauterize, coagulate/desiccate, and/or simply reduce or slow bleeding simply by controlling the intensity, frequency and duration of the electrosurgical energy applied through the jaw members to the tissue.

In order to effectively seal vessels (or tissue) two predominant mechanical parameters must be accurately controlled—the pressure applied to the vessel (tissue) and the gap distance between the electrodes—both of which are affected by the thickness of the sealed vessel. The pressure applied to the vessel may be fixed by the mechanical design of the instrument.

It can be difficult for surgeons to visually determine the gap distance between electrodes before energy application. After energy application, it may be difficult to ensure that the jaws of the forceps have achieved an appropriate seal closure. The visualization of the surgical field may be difficult because of blood within the surgical field, lack of complete vessel (tissue) dissection, or isolation.

Some electrosurgical forceps mechanically limit the jaw aperture (maximum jaw opening) to prevent the forceps from being used on vessels or tissue beyond the forceps limits. However, such mechanical limits may prevent the electrosurgical forceps from being used as a multi-purpose instrument. Particularly, limiting the jaw aperture would prevent the electrosurgical forceps from functioning as a grasper and/or a dissecting instrument.

### SUMMARY

In an aspect of the present disclosure, a surgical instrument includes a housing, a shaft, an end effector, and a sensor. The shaft extends distally from the housing. The end effector is disposed at a distal end of the shaft and includes first and second jaw members that are moveable relative to one another from a first configuration to a second configuration. In the first configuration, the first and second jaw members are spaced relative to one another and in the second configuration, the first and second jaw members are closer to one another for approximating tissue. The first and second jaw members define a gap distance therebetween. The sensor is positioned within the housing and operable to determine the size of the gap distance. The first and second jaw members configured to be electrical activated to treat tissue between the first and second jaw members when the size of the gap distance is within an acceptable range.

In some aspects, the elongated shaft defines a longitudinal axis and is translatable along the longitudinal axis to move the first and second jaw members between the first and second configurations. A proximal end of the elongated shaft may be longitudinally translatable within the housing and the sensor may be positioned adjacent the proximal end of the elongated shaft. The sensor may detect the position of the proximal end of the elongated shaft relative to the housing to determine the size of the gap distance.

In certain aspects, the surgical instrument includes a drive assembly that is disposed within the housing and that is operatively associated with moveable handle to longitudinal translate the elongated shaft. The drive assembly may include a tube that has a distal end coupled to the distal end of the elongated shaft and a proximal end that is positioned proximal to the moveable handle. The sensor may be positioned adjacent the proximal end of the tube to detect the position of the proximal end of the tube relative to the housing for determining the size of the gap distance.

In particular aspects, the surgical instrument includes a drive rod that is slidably disposed within the elongated shaft. A proximal end of the drive rod may extend into the housing. The drive rod may define a longitudinal axis and may be translatable along the longitudinal axis to move the first and second jaw members between the first and second configurations. The sensor may be positioned adjacent the proximal end of the drive rod. The sensor may detect the position of the proximal end of the drive rod relative to the housing to determine the size of the gap distance.

In some aspects, the surgical instrument includes a moveable handle that is operatively associated with the drive rod to longitudinally translate the drive rod. The drive rod may include a proximal drive plate and a distal drive plate with the moveable handle positioned between the proximal and distal drive plates. The sensor may be positioned adjacent the proximal drive plate for detecting the position of the proximal drive plate relative to the housing to determine the size of the gap distance. Additionally or alternatively, the sensor may be positioned adjacent the distal drive plate for detecting the position of the distal drive plate relative to the housing to determine the size of the gap distance.

In certain aspects, the sensor is configured to provide feedback of the size of the gap distance. The first and second jaw members may be configured to deliver electrosurgical energy to tissue between the first and second jaw members. The sensor may be configured to provide feedback when the size of the gap distance is suitable for applying electrosurgical energy to tissue. The sensor may be optical, magnetic, inductive, mechanical, or any combination thereof.

In another aspect of the present disclosure, a surgical instrument includes a first member, a second member, a pivot, a flag, and a sensor. The first member has proximal and distal end portions with the distal end portion including a first jaw member. The second jaw member has proximal and distal end portions with the distal end portion including a second jaw member. The first and second jaw members define a gap distance therebetween. The pivot passes through the first and second members between the respective proximal and distal end portions such that the first and second members are pivotable relative to one another to pivot the first and second jaw members between first and second configurations. In the first configuration, the first and second jaw members are spaced relative to one another and in the second configuration, the first and second jaw members are closer to one another for approximating tissue. The flag has a distal end that is coupled to the distal end portion of the second member and extends proximally along the second member to a free end that is positioned adjacent the proximal end portion of the second member. The sensor is positioned adjacent the free end of the flag for determining the size of the gap distance. If the size of the gap distance is within an acceptable range, the first and second jaw members may be electrically activated to treat tissue approximated between the first and second jaw members.

In some aspects, the sensor is disposed on the proximal end portion of the second member. The sensor may detect the position of the free end of the flag to determine the size of the gap distance between the first and second members. The second member may be flexible such that flexation of the second member is indicative of the size of the gap distance. The sensor may be configured to detect the flexation of the second member to determine the size of the gap distance.

In another aspect of the present disclosure, a method of determining the size of a gap distance of a surgical instrument includes positioning the first and second jaw members of the surgical instrument over tissue such that the tissue is positioned between the first and second jaw members, determining the size of the gap distance with a sensor positioned remote to the first and second jaw members, and activating the first and second jaw members to deliver electrosurgical energy to the tissue positioned between the first and second jaw members when the size of the gap distance in in an acceptable range.

In some aspects, the method may include pivoting a first handle of the surgical instrument towards a second handle of the surgical instrument to move the first and second jaw members towards one another. Determining the size of the gap distance may include determining the flexation of the second handle with the sensor. Determining the flexation of the second handle with the sensor may include detecting the position of a free end of a flag relative to the second handle. The free end of the flag may extend from a distal end of the flag which is fixed to the second jaw member.

In certain aspects, determining the size of the gap distance includes detecting the position of a proximal end of a shaft relative to a housing of the surgical instrument with the sensor. The shaft may extend distally from the housing with the first and second jaw members positioned at a distal end of the shaft. The sensor may be positioned within the housing.

In particular aspects, determining the size of the gap distance includes detecting the position of a drive rod within a housing of the surgical instrument. The drive rod may extend through an elongated shaft and be slidable within the elongated shaft which extends distally from the housing. The first and second jaw members may be positioned at a distal end of the elongated shaft. Detecting the position of the drive rod within the housing of the surgical instrument may include detecting a position of a proximal end of the drive rod relative to the housing with the sensor. The sensor may be positioned within the housing adjacent the proximal end of the drive rod.

In some aspects, the method includes moving a moveable handle from an initial position to an approximated position to longitudinally translate the drive rod within the elongated shaft. The moveable handle may be positioned between proximal and distal drive plates of the drive rod. Detecting the position of the drive rod within the housing of the surgical instrument includes detecting the position of the proximal drive plate of the drive rod relative to the housing with the sensor. The sensor may be positioned within the housing adjacent the proximal drive plate. Additionally or alternatively, detecting the position of the drive rod within the housing of the surgical instrument may include detecting the position of the distal drive plate of the drive rod relative to the housing with the sensor. The sensor may be positioned adjacent the distal drive plate.

In certain aspects, the method includes providing feedback when the size of the gap distance is suitable for delivering electrosurgical energy to the tissue between the first and second jaw members of the surgical instrument. The sensor may detect the position of a component of the surgical instrument from a position that is remote to the first and second jaw members to determine the size of the gap distance optically, magnetically, inductively, mechanically, or any combination thereof.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of an endoscopic electrosurgical forceps in accordance with the present disclosure;

FIG. 2 is a side, cut-away view of the endoscopic electrosurgical forceps of FIG. 1 showing a pair of jaw members in an open configuration with a clamping handle in an initial position;

FIG. 3 is a side, cut-away view of the endoscopic electrosurgical forceps of FIG. 1 in an activatable configuration with the clamping handle in an approximated position;

FIG. 4 is a side, cut-away view of the endoscopic electrosurgical forceps of FIG. 1 with the clamping handle in the approximated position and jaw members closed about tissue within an unacceptable jaw aperture range;

FIG. 5 is a side, cut-away view of another endoscopic electrosurgical forceps in accordance with the present disclosure in an open configuration with a clamping handle in an initial position;

FIG. 6 is a side, cut-away view of the endoscopic electrosurgical forceps of FIG. 5 in an activatable configuration with the clamping handle in an approximated position and jaw members closed about tissue within an acceptable jaw aperture range;

FIG. 7 is a side, cut-away view of the endoscopic electrosurgical forceps of FIG. 5 with the clamping handle in the approximated position and jaw members closed about tissue within an unacceptable jaw aperture range;

FIG. 8 is a side view of an open electrosurgical forceps in accordance with the present disclosure with handles in an initial position and jaw members in an open position;

FIG. 9 is a side view of the open electrosurgical forceps of FIG. 8 in an activatable configuration with the handles in an approximated position and jaw members closed about tissue within appropriate jaw aperture range;

FIG. 10 is a side view of the open electrosurgical forceps of FIG. 8 in an open configuration with the clamping handle in the approximated position and jaw members closed about tissue within an unacceptable jaw aperture range; and

FIG. 11 is a schematic view of a robotic surgical system in accordance with the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician.

This disclosure relates generally to position sensors positioned in or on the body of an electrosurgical forceps to determine a gap distance or jaw aperture defined between jaw members of the electrosurgical forceps. The position sensor may sense the position of a closure tube that correlates to the jaw aperture, may sense the position of a push rod, or may sense the position or angle of a flag attached adjacent a distal end of the electrosurgical forceps to determine the jaw aperture. The position sensor may also provide feedback (e.g., audible, tactile, or visual) to a clinician when the gap distance between jaw members is in an acceptable range suitable for sealing tissue between the jaw members with electrosurgical energy. The acceptable range for the gap distance to seal tissue is about 0.001 inches to about 0.006 inches. In addition, the position sensor may be in communication with an energy activation circuit to prevent delivery of electrosurgical energy when the gap distance is beyond a predetermined limit (i.e., above the acceptable range).

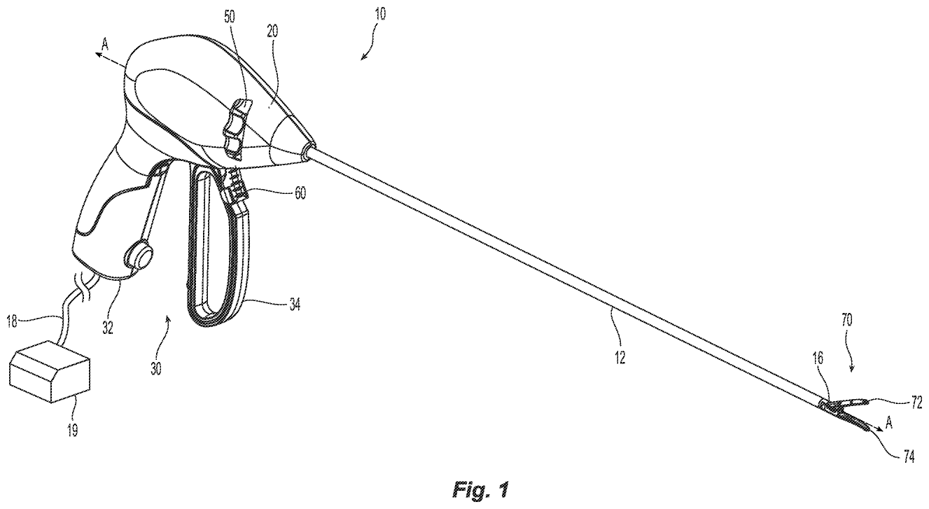

Referring now to FIG. 1, an endoscopic electrosurgical forceps 10 is provided in accordance with the present disclosure and includes a housing 20, a handle assembly 30, a rotating assembly 50, a trigger assembly 60, and an end effector assembly 70. The end effector assembly 70 includes first and second jaw members 72, 74 for grasping, sealing, and treating tubular vessels and vascular tissue. For the purposes herein, forceps 10 will be described generally. However, the various particular aspects of this particular forceps are detailed in U.S. Patent Publication No. 2014/0257274, the entire contents of which are incorporated by reference herein.

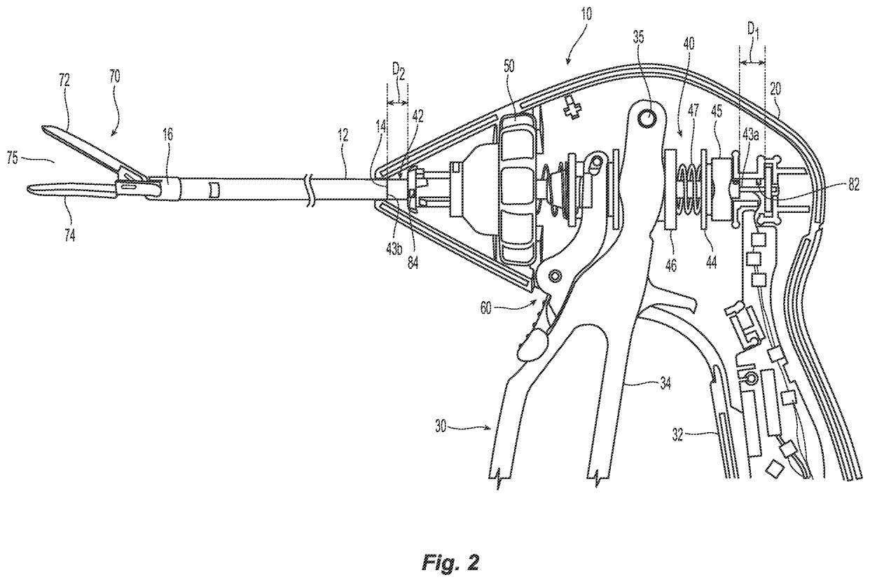

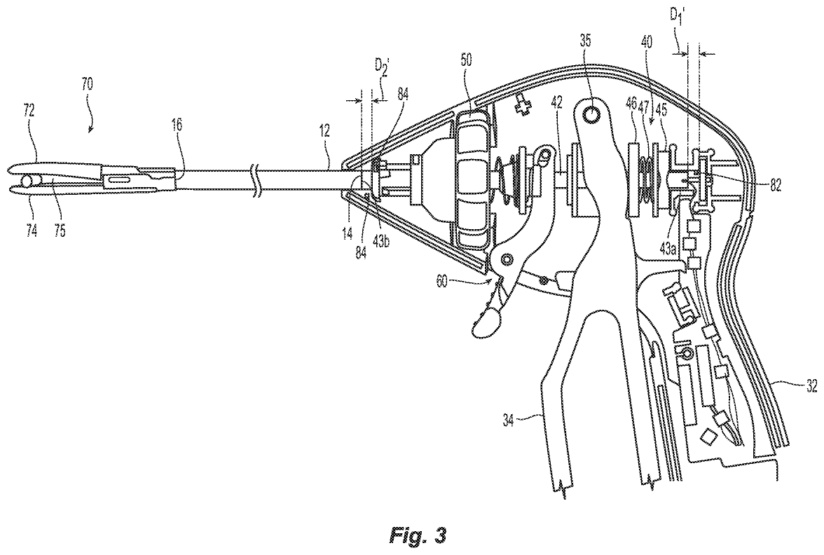

The forceps 10 includes a shaft 12 that defines a longitudinal axis “A-A” of the forceps 10 and has a proximal end 14 (FIG. 2) and a distal end 16. The proximal end 14 of the shaft 12 is operatively engaged to the housing 20. The distal end 16 of the shaft 12 is configured to mechanically engage the end effector assembly 70 to move the first and second jaw members 72, 74 between an open configuration (FIG. 2) and a closed configuration (FIG. 3) as detailed below. Once closed, the gap distance between the jaw members 72, 74 will determine if the forceps 10 can be energized.

Forceps 10 also includes an electrosurgical cable 18 that connects the forceps 10 to a source of electrosurgical energy, e.g., a generator 19. The source of electrosurgical energy provides electrosurgical energy to the end effector 70 of the forceps 10. It is also contemplated that the forceps 10 may include an energy source (e.g., a battery (not shown)) and an electrosurgical generator (not shown) positioned on or within the housing 20 to provide electrosurgical energy to the forceps 10.

Referring to FIG. 2, the handle assembly 30 includes a fixed handle 32 and a moveable handle 34. The fixed handle 32 is integrally associated with housing 20 and the moveable handle 34 is movable relative to the fixed handle 32 to translate the shaft 12 along the longitudinal axis “A-A”. The rotating assembly 50 is disposed substantially within the housing 20 and is rotatable approximately 180 degrees in either direction about the longitudinal axis “A-A” to rotate the end effector assembly 70 relative to the housing 20.

The moveable handle 34 has an upper end 35 that is pivotally secured within the housing 20 and engaged with a drive assembly 40 of the forceps 10. The drive assembly 40 includes a tube 42, a proximal drive plate 44, and a distal drive plate 46. The tube 42 has a proximal end 43a that passes through the proximal and distal drive plates 44, 46 and a distal end 43b that passes through the rotating assembly 50. The distal end 43b of the tube 42 is coupled to the proximal end 14 of the shaft 12 to translate the shaft 12 along the longitudinal axis “A-A”.

The proximal drive plate 44 is coupled to the proximal end 43a of the tube 42 to translate the tube 42 along the longitudinal axis “A-A”. A first biasing member 45 is positioned between the proximal drive plate 44 and the housing 20 to urge the proximal drive plate 44 distally such that the first and second jaw members 72, 74 of the end effector assembly 70 are biased in the open configuration (FIG. 2). A second biasing member 47 is positioned between the proximal and distal drive plates 44, 46 to urge distal drive plate 46 away from the proximal drive plate 44. The distal drive plate 46 engages the moveable handle 34 to move the moveable handle 34 towards its initial position.

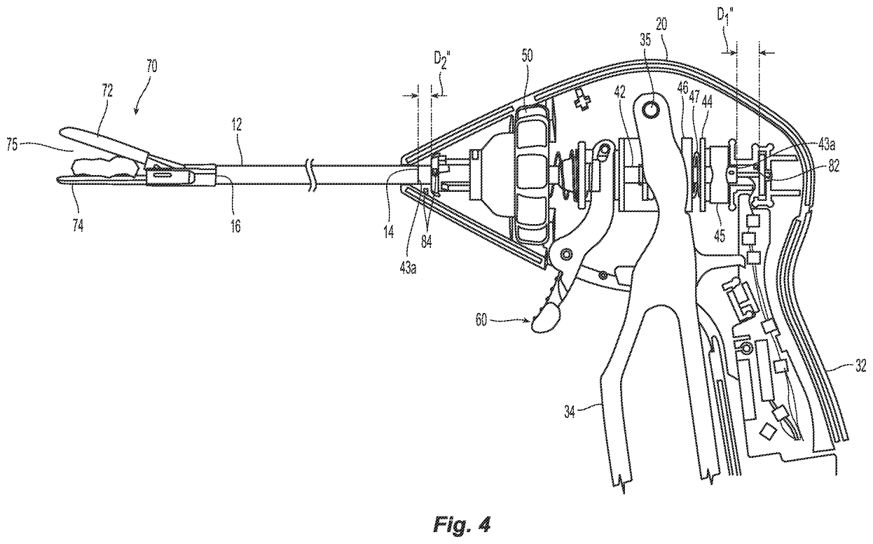

With additional reference to FIGS. 3 and 4, as the moveable handle 34 is pivoted towards its approximated or closed position, the moveable handle 34 moves the distal drive plate 46 distally against the second biasing member 47. As the distal drive plate 46 is moved distally, the second biasing member 47 applies a handle force to the proximal drive plate 44 to urge the proximal drive plate 44 proximally. Proximal movement of the proximal drive plate 44 is resisted by a clamping force which is the combination of a compression force of the first biasing member 45 and a closure force of the first and second jaw members 72, 74 of the end effector assembly 70. The closure force is the force exerted on the first and second jaw members 72, 74 by tissue positioned within the jaw aperture 75 as detailed below. When the handle force is greater than the clamping force, the proximal drive plate 44 is moved distally such that the first and second jaw members 72, 74 are moved towards the activatable configuration and the first biasing member 45 is compressed as shown in FIG. 3. As a result, the gap distance is within an acceptable range for sealing tissue between the first and second jaw members 72, 74 with electrosurgical energy and the first and second jaw members 72, 74 can then be selectively energized. When the handle force is less than the clamping force, the proximal drive plate 44 resists proximal movement such that the second biasing member 47 is compressed between the proximal and distal drive plates 45, 47 as shown in FIG. 4. As a result, the gap distance is unacceptable or outside of the acceptable range and the first and second jaw members 72, 74 are prevented from energizing.

The first and second biasing members 45, 47 are calibrated to limit closure force of the first and second jaw members 72, 74 of the end effector assembly 70. When a small vessel, or amount of tissue, or a large compressible vessel, or amount of tissue, is positioned within a jaw aperture 75 (i.e., between the first and second jaw members 72, 74), a closure force of the first and second jaw members 72, 74 is small such that the handle force is greater than or equal to the clamping force to allow the first and second jaw members 72, 74 to move to an activatable configuration as shown in FIG. 3. In an activatable configuration, the gap distance is in the acceptable range for sealing tissue within the jaw aperture 75. When a large vessel, or amount of tissue, is positioned within a jaw aperture 75 (between the jaw members 72, 74), the closure force of the first and second jaw members 72, 74 is large such that the handle force is less the clamping force. When the handle force is less than the clamping force, the first and second jaw members 72, 74 resist moving to an activatable configuration and remain substantially in the open configuration as shown in FIG. 4. In such an open configuration, the gap distance of between the jaw members 72, 74 is outside of the acceptable range for sealing the vessel, or tissue, within the jaw aperture 75.

With reference to FIGS. 2-4, the forceps 10 includes first and second position sensors 82, 84 for detecting the position of the tube 42 to determine the gap distance between the jaw members 72, 74. The first and second position sensors 82, 84 are positioned within the housing 20 remote to the end effector 70. The first position sensor 82 is positioned adjacent the proximal end 43a of the tube 42 to detect the position of the proximal end 43a relative to the housing 20. The second position sensor 84 is positioned adjacent the distal end 43b of the tube 42 to detect the position of the distal end 43b relative to the housing 20. In a fully open configuration of the first and second jaw members 72, 74 (FIG. 2), the tube 42 is in a distal-most position such that a detected distance D₁ between the first sensor 82 and the proximal end 43a of the tube 42 and a detected distance D₂ between the second sensor 84 and the distal end 43b of the tube 42 are at a maximum value. In an activatable configuration of the first and second jaw members 72, 74 (FIG. 3), the tube 42 is in an activatable position, proximal of its distal-most position, such that a detected distance D₁′ between the first sensor 82 and the proximal end 43a of the tube 42 and a detected distance D₂′ between the second sensor 84 and the distal end 43b of the tube 42 are in an activatable range of values which is less than the maximum value. When the first and second jaw members 72, 74 are in an activatable configuration, the first and second sensors 82, 84 may provide feedback to a clinician that the gap distance is within the acceptable range that is suitable for sealing tissue between the first and second jaw members 72, 74 with electrosurgical energy. This feedback may be audible, visual, or tactile.

With particular reference to FIG. 4, when a large vessel, or amount of tissue, is positioned within the jaw aperture 75 and the moveable handle 34 is in the approximated or closed position, the tube 42 is between the distal-most position and an activatable position such that the gap distance is beyond or outside of the acceptable range for sealing tissue. In such an open position, a detected distance D₁″ between the first sensor 82 and the proximal end 43a of the tube 42 and a detected distance D₂″ between the second sensor 84 and the distal end 43b of the tube 42 is between the maximum value and an activatable range values. When the moveable handle 34 reaches the approximated or closed position and the first and second jaw members 72, 74 are between the fully open configuration and the activatable configuration, the forceps 10 may provide feedback to a clinician that the gap distance between the jaw members 72, 74 is outside of the acceptable range for sealing tissue. This feedback may be audible, visual, or tactile.

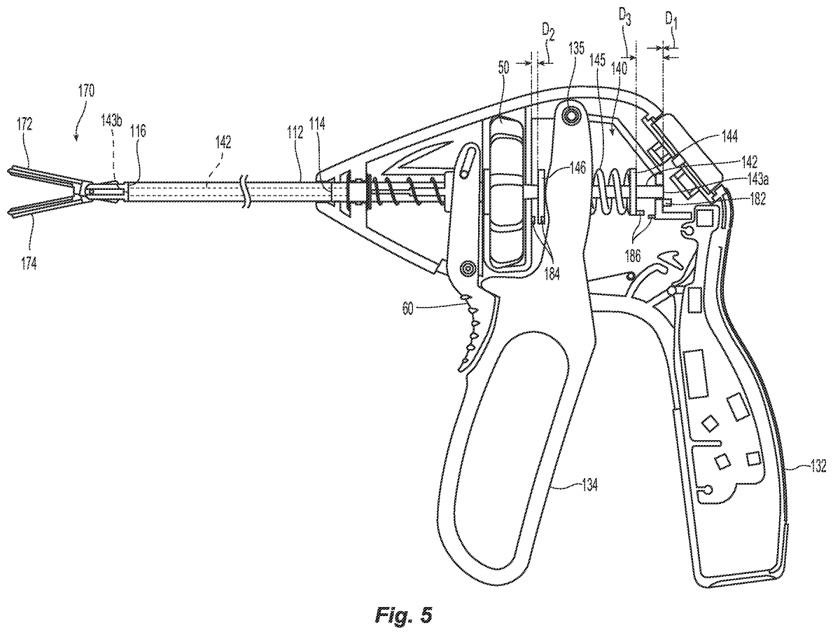

Referring now to FIGS. 5-7, another endoscopic electrosurgical forceps 110 is provided in accordance with the present disclosure. The forceps 110 is substantially similar to forceps 10 detailed above, as such only the differences will be detailed herein. For reasons of brevity, elements of the endoscopic forceps 110 similar to elements of the endoscopic forceps 10 are identified with similar labels with a “1” preceding the previous label. For the purposes herein, forceps 110 will be described generally. However, the various particular aspects of this particular forceps are detailed in U.S. Patent Publication Nos. 2013/0296848 and 2013/0296922, the entire contents of each of these disclosures is incorporated by reference herein.

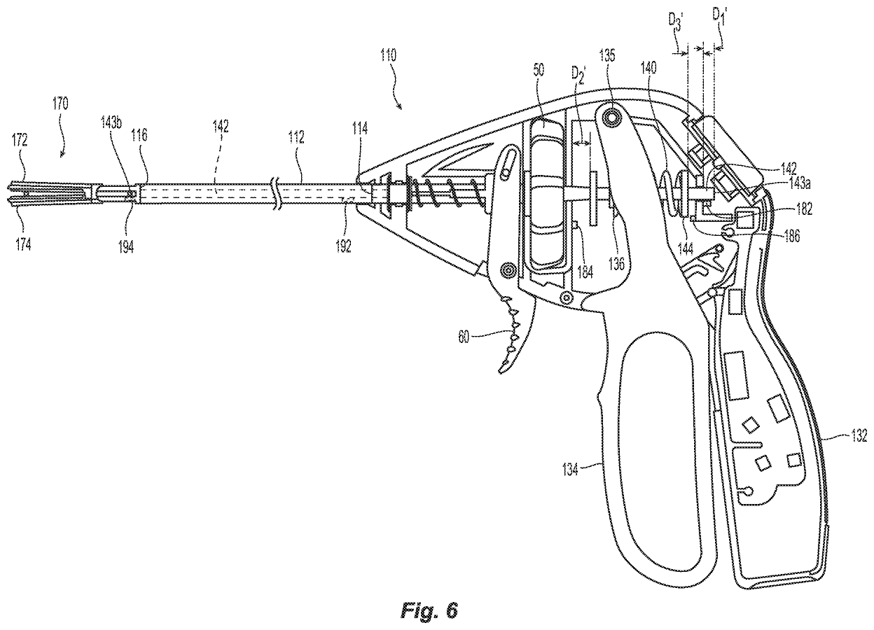

A drive assembly 140 of the forceps 110 includes a drive rod 142, a proximal drive plate 144, and a distal drive plate 146. The drive rod 142 is translatable along the longitudinal axis “A-A” defined by a shaft 112 to move first and second jaw members 172, 174 of an end effector assembly 170 between an open configuration (FIG. 5) and a closed configuration (FIG. 6). Once closed, the gap distance between the jaw members 172, 174 will determine if the forceps 110 can be energized. The drive rod 142 extends from a housing 120 of the forceps 110, through the shaft 112, and to the end effector assembly 170. The drive rod 142 includes a proximal end 143a that is disposed within the housing 120 of the forceps 110 and a distal end 143a that is operatively associated with the end effector assembly 170.

The proximal drive plate 144 is coupled to the drive rod 142 adjacent the proximal end 143a of the drive rod 142. The distal drive plate 146 is coupled to the drive rod 142 distal of the proximal drive plate 144. A moveable handle 134 includes a plunger 136 (FIG. 6) that is positioned about the drive rod 142 between the proximal and distal drive plates 144, 146. A biasing member 145 is positioned about the drive rod 142 between the plunger 136 and the proximal drive plate 144 to urge the moveable handle 134 towards an initial position (FIG. 5). In the initial position, the moveable handle 134 may engage the distal drive plate 146 to urge drive rod 142 distally such that the first and second jaw members 172, 174 of the end effector assembly 170 are moved towards the fully open configuration.

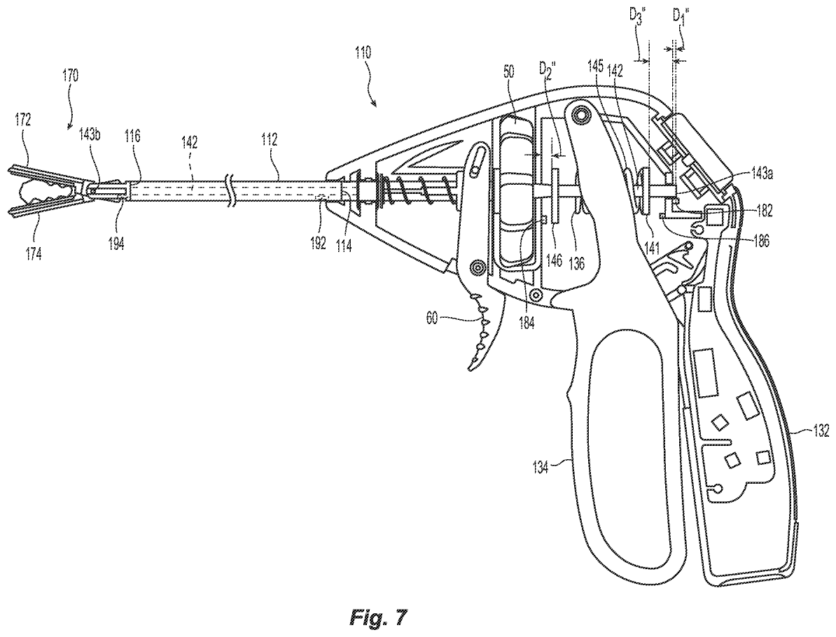

As the moveable handle 134 is pivoted towards an approximated or closed position (FIGS. 6 and 7), the plunger 136 moves proximally within the housing 120. As the plunger 136 moves proximally, the plunger 136 exerts a handle force on the biasing member 145 which exerts a clamping force on the proximal drive plate 144. In response, the proximal drive plate 144 translates the drive rod 142 proximally. As the drive rod 142 translates proximally, the distal end 143b of the drive rod 142 moves the first and second jaw members 172, 174 towards the closed configuration and, particularly, an activatable configuration such that the gap distance between the jaw members 172, 174 is within the acceptable range.

As gap distance is reduced, the first and second jaw members 172, 174 engage a vessel or tissue positioned therebetween. As the first and second jaw members 172, 174 engage a vessel or tissue, the vessel or tissue resists movement of the first and second jaw members 172, 174 towards an activatable configuration such that a closure force is required to move the first and second jaw members 172, 174 towards the activatable configuration. The clamping force is the sum of the closure force exerted by tissue positioned within the jaw aperture 175 and mechanical resistance to moving the first and second jaw members 172, 174 towards an activatable configuration. If the clamping force is less than the handle force, the proximal drive plate 144 is moved proximally to translate the drive rod 142 proximally and to move the first and second jaw members 172, 174 towards an activatable configuration until the moveable handle 134 is in the approximated or closed position and the first and second jaw members 172, 174 are in an activatable configuration as shown in FIG. 6. If the clamping force is greater than the handle force, the handle force compresses the biasing member 145. The moveable handle 134 is moved towards the approximated or closed position increasing the handle force as the biasing member 145 is compressed until the handle force exceeds the clamping force or the moveable handle 134 reaches its approximated or closed position without the first and second jaw members 172, 174 reaching the activatable configuration as shown in FIG. 7. As detailed above, in the activatable configuration of the first and second jaw members 172, 174 the gap distance between the jaw members 172, 174 is within the acceptable range for sealing a vessel or tissue within the jaw aperture 175 with electrosurgical energy.

With continued reference to FIGS. 5-7, the forceps 110 includes first, second, and third position sensors 182, 184, 186 for detecting the position of the drive rod 142 relative to the housing 120 to determine the gap distance. The first position sensor 182 is positioned within the housing 120 adjacent the proximal end 143a of the drive rod 142 to detect the position of the proximal end 143a relative to the housing 120 as indicated by a detected distance D₁. Alternatively, the first position sensor 182 may be disposed on the distal end 143a of the drive rod to detect the position of the proximal end 143a relative to the housing 120 as indicated by the detected distance D₁. The second position sensor 184 is positioned on the housing adjacent the distal drive plate 146 to detect the position of the distal drive plate 146 relative to the housing 120 as indicated by a detected distance D₂. Alternatively, the second position sensor 184 may be disposed on the distal drive plate 146 to detect the position of the distal drive plate 146 relative to the housing 120 as indicated by the detected distance D₂. The third position sensor 186 is positioned on the proximal drive plate 144 to detect the position of the proximal drive plate 144 relative to the housing 120 as indicated by a detected distance D₃.

In a fully open configuration of the first and second jaw members 172, 174 (FIG. 5), the drive rod 142 is in a distal-most position relative to the housing 120 such that the detected distances D₁ and D₂ have a minimum value and detected distance D₃ has a maximum value. In the activatable configuration of the first and second jaw members 172, 174 (FIG. 6), the drive rod 142 is in an activatable position, proximal of its distal-most position, such that detected distances D₁′ and D₂′ are in a range of activatable values greater than the minimum value and the detected distance D₃′ is in a range of activatable values less than the maximum value. When the first and second jaw members 172, 174 are in an activatable configuration, the first, second, and third position sensors 182, 184, 186 may provide feedback to a clinician that the gap distance of the jaw aperture 175 is within an acceptable range for sealing tissue between the first and second jaw members 172, 174. This feedback may be audible, visual, or tactile.

With particular reference to FIG. 7, when a large vessel, or amount of tissue, is positioned within the jaw aperture 75 and the moveable handle 134 is in the approximated or closed position, the drive rod 142 is between the distal-most position and the activatable position such that the gap distance is not suitable for application of electrosurgical energy. In such a position, the detected distances D₁″ and D₂″ are between the minimum value and the range of activatable values and the detected distance D₃″ is between the maximum value and the range of activatable values. When the moveable handle 134 reaches the approximated or closed position and the first and second jaw members 172, 174 define a gap distance outside of the acceptable range, the forceps 110 may provide feedback to a clinician that the gap distance is not suitable for sealing tissue. This feedback may be audible, visual, or tactile.

With reference to FIGS. 6 and 7, position sensors may be positioned in various locations about the forceps 110 to detect a position of a moveable structure relative to a fixed structure to determine the gap distance of the first and second jaw members 172, 174. For example, a position sensor 192 may be positioned within the shaft 112 to detect the position of the drive rod 142 relative to the shaft 112 to determine the gap distance. In another example, a position sensor 194 may be positioned adjacent a distal end 116 of the shaft 112 to detect the position of the distal end 143b of the drive rod 142 to determine the gap distance.

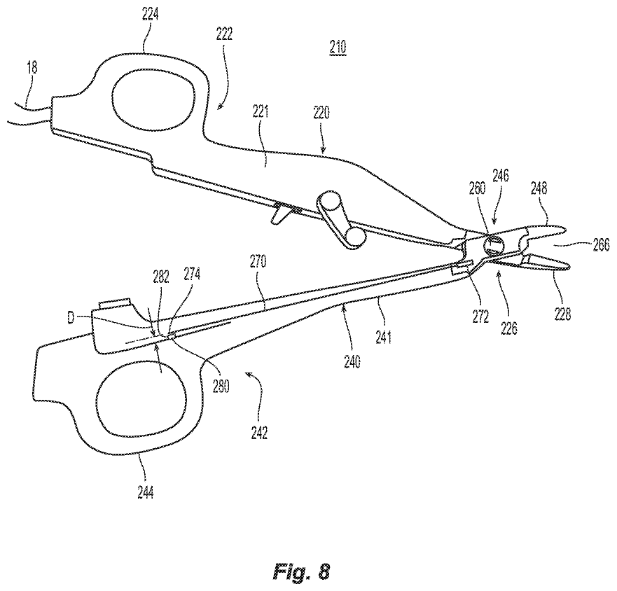

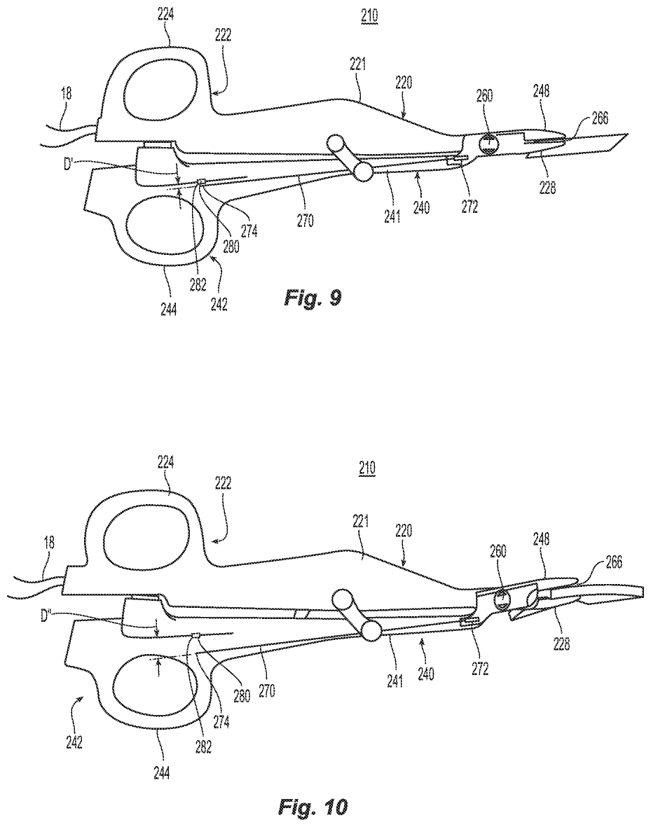

Referring now to FIGS. 8-10, an open electrosurgical forceps 210 is provided in accordance with the present disclosure and includes a first member 220 and a second member 240. Each of the first and second member 220, 240 includes a shaft 221, 241 having respective proximal end portions 222, 242 and respective distal end portions 226, 246. Each proximal end portion 222, 242 includes a handle 224, 244 and each distal end portion 226, 246 includes a jaw member 228, 248. The shafts 221, 241 are pivotable relative to one another about a pivot 260 to pivot the jaw members 228, 248 between an open configuration (FIG. 8) and a closed configuration (FIG. 9). For the purposes herein, forceps 210 will be described generally. However, the various particular aspects of this particular forceps are detailed in U.S. Patent Publication No. 2012/0083827, the entire contents of which are incorporated by reference herein.

The pivot 260 passes through the shafts 221, 241 between the handles 224, 244 and the jaw members 228, 248. The first member 220 includes an electrosurgical cable 18 that connects the forceps 210 to a source of electrosurgical energy. Additionally or alternatively, the second member 240 may include an electrosurgical cable (not shown) that connects the forceps 210 to a source of electrosurgical energy.

The shafts 221, 241 are designed to transmit a particular closure force to the jaw members 228, 248 as the shafts 221, 241 are pivoted towards the closed configuration. In particular, the shafts 221, 241 effectively act together in a spring-like manner (i.e., bending that behaves like a spring) such that the length, width, height, and deflection of the shafts 221, 241 directly effects the closure force imposed by jaw members 228, 248. The jaws 228 and 248 are more rigid than the shafts 221, 241 such that strain energy stored in the shafts 221, 241 provides a constant closure force between the jaw members 228, 248 in response to a handle force applied to the handles 224, 244.

With additional reference to FIG. 9, as the handles 224, 244 are moved towards each other, the shafts 221, 241 pivot about the pivot 260 such that the jaw members 228, 248 move towards the closed configuration. As the jaw members 228, 248 move towards the closed configuration with a small vessel, or amount of tissue, or a large compressible vessel, or amount of tissue, positioned within a jaw aperture 266, the jaw members 228, 248 impose a closure force to the vessel, or tissue. If the closure force required to move the jaw members 228, 248 towards the closed configuration is less than or equal to the handle force applied to the handles 224, 244, the jaw members 228, 248 move towards the closed configuration effecting compression of the vessel, or tissue, until the handles 224, 244 are in an approximated or closed position and the jaw members 228, 248 are in an activatable configuration as shown in FIG. 9. In an activatable configuration, the gap distance between the jaw members 228, 248 is in an acceptable range for sealing tissue within the jaw aperture 266. If the closure force required to move the jaw members 228, 248 is greater than the handle force applied to the handles 224, 244, the shafts 221, 241 flex or bend towards one another until the handles 224, 244 are in the approximated or closed position with the jaw members 228, 248 remaining in a generally open configuration (e.g., between an open configuration and an acceptable activatable configuration) as shown in FIG. 10. In such a configuration, the gap distance between the jaw members 228, 248 is outside of the acceptable range for sealing the vessel, or tissue, within the jaw aperture 266.

Continuing to refer to FIGS. 8-10, the forceps 210 includes a deflection flag 270 attached at a fixed end 272 to the distal end portion 246 of the shaft 241. The deflection flag 270 extends from the fixed end 272 along the shaft 241 to a free end 274 positioned adjacent the proximal end portion 242 of the second member 240. The proximal end portion 242 is moveable relative to the free end 274 as the shaft 241 flexes or bends in response to the handle force and the closure force.

When the handles 224, 244 are in the approximated or closed position, the amount of flexation of the shaft 241 correlates to the gap distance between the jaw members 228, 248. The flexation of the shaft 241 is measurable by determining a change in a distance D between a fixed point 280 on the proximal end portion 242 of the second member 240 and the free end 274 of the flag 270 in the open configuration and a distance D′ or D″ between the fixed point 280 and the free end 274 in the approximated configuration.

As shown in FIG. 8, the handles 224, 244 are in a fully open position such that the shaft 241 of the second member 240 is unflexed (i.e., substantially straight) and the free end 274 of the flag 270 defines a distance D with the fixed point 280 in a first direction. When the handles 224, 244 are moved to the approximated or closed position with an appropriate amount tissue positioned within the jaw aperture 266, the shaft 241 of the second member 240 flexes such that a distance D′ is defined between the fixed point 280 and the free end 274 when the jaw members 228, 248. As shown in FIG. 9, in an activatable configuration of the jaw members 228, 248, the free end 274 and the fixed point 280 are substantially aligned.

When a large amount of tissue is positioned within the jaw aperture 266, the shaft 241 of the second member 240 flexes as the handles 224, 244 are moved to the approximated or closed position and the jaw members 228, 248 remain in a generally open configuration such that a distance D″ is defined between the fixed point 280 and the free end 274 in response to the handle force and the closure force as shown in FIG. 10. As shown, when the closure force is greater than the handle force, the jaw members 228, 248 remain in generally open. In such instances as shown in FIG. 10, the change from the distance D to the distance D″ is larger than the change from the distance D to the distance D′ and the gap distance between the jaw members 228, 248 is unacceptable resulting in a non-activatable configuration.

Similar to the forceps 10 and 110 detailed above, when the jaw members 228, 248 are in an activatable configuration (FIG. 9), the gap distance between the jaw members 228, 248 is outside of the acceptable range for sealing tissue. As shown in FIG. 10, the gap distance between the jaw members 228, 248 is outside of the acceptable range for sealing tissue.

Continuing to refer to FIGS. 8-10, the forceps 210 includes a position sensor 282 for detecting the distance between the fixed point 280 and the free end 274 (e.g., D, D′, and D″). The position sensor 282 is positioned remote to the jaw members 228, 248. The position sensor 282 is disposed on the second member 240 adjacent the fixed point 280 to detect the position of the free end 274 relative to the fixed point 280 to determine the flexation of the shaft 241 of the second member 240. From the flexation of the shaft 241 of the second member 240, the gap distance is determined. As shown, the position sensor 282 is positioned on the second member 240 adjacent the fixed point 280; however, the position sensor 282 may be positioned on the free end 274 and be configured to detect the position of the fixed point 280 relative to the free end 274.

As shown, the deflection flag 270 is substantially linear between the first and second ends 272, 274; however, the deflection flag 270 may be curved or have non-linear portion between the fixed end 272 and the free end 274.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

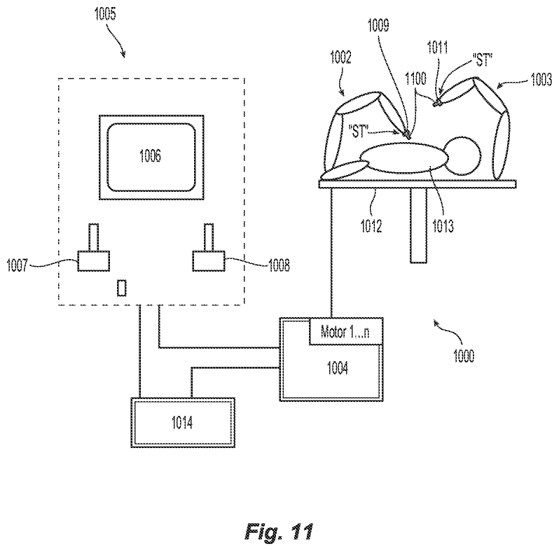

Referring initially to FIG. 11, a medical work station is shown generally as work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus the surgical tool (including end effector 1100) execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of end effector 1100. Medical work station 1000 may also include more than two robot arms 1002, 1003, the additional robot arms likewise being connected to control device 1004 and being telemanipulatable by means of operating console 1005. A medical instrument or surgical tool (including an end effector 1100) may also be attached to the additional robot arm. Medical work station 1000 may include a database 1014, in particular coupled to with control device 1004, in which are stored, for example, pre-operative data from patient/living being 1013 and/or anatomical atlases.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

1-20. (canceled)

21. A surgical instrument, comprising:
a housing supporting a movable handle;
an elongated shaft defining a longitudinal axis and extending distally from the housing, the elongated shaft configured to be translated along the longitudinal axis by the movable handle, the elongated shaft defining a distal end and a proximal end positioned within the housing;
an end effector disposed at the distal end of the elongated shaft and including first and second jaw members, the first and second jaw members defining a gap distance therebetween;
a sensor coupled to the housing, the sensor positioned proximal of the proximal end of the elongated shaft and longitudinally aligned with the proximal end of the elongated shaft along the longitudinal axis, the sensor configured to detect a position of the proximal end of the elongated shaft relative to the housing to determine the size of the gap distance, wherein, if the size of the gap distance is within a predetermined range, the first and second jaw members are configured to be electrically activated to treat tissue between the first and second jaw members.

22. The surgical instrument according to claim 21, wherein the elongated shaft is translatable along the longitudinal axis to move the first and second jaw members relative to one another from a first configuration wherein the first and second jaw members are spaced relative to one another, to a second configuration wherein the first and second jaw members are closer to one another for approximating tissue.

23. The surgical instrument according to claim 20, wherein the proximal end of the elongated shaft is positioned proximal of the movable handle.

24. The surgical instrument according to claim 20, further including a drive assembly disposed within the housing and operatively associated with the moveable handle to longitudinally translate the elongated shaft.

25. The surgical instrument according to claim 20, wherein the drive assembly includes a tube having a distal end coupled to the proximal end of the elongated shaft and a proximal end longitudinally aligned with the sensor along the longitudinal axis.

26. The surgical instrument according to claim 25, wherein the drive assembly includes a biasing member configured to bias the elongated shaft distally.

27. The surgical instrument according to claim 21, wherein the sensor is configured to provide feedback of the size of the gap distance.

28. The surgical instrument according to claim 21, wherein the first and second jaw members are configured to deliver electrosurgical energy to tissue between the first and second jaw members, and wherein the sensor is configured to provide feedback when the size of the gap distance is suitable for applying electrosurgical energy to tissue.

29. The surgical instrument according to claim 21, wherein the sensor is at least one of optical, magnetic, inductive, or mechanical.

30. The surgical instrument according to claim 21, wherein the sensor is fixedly coupled to the housing.

31. A surgical instrument, comprising:
a housing supporting a movable handle;
an elongated shaft defining a longitudinal axis and extending distally from the housing, the elongated shaft configured to be translated along the longitudinal axis by the movable handle, the elongated shaft defining a distal end and a proximal end positioned within the housing;
an end effector disposed at the distal end of the elongated shaft and including first and second jaw members, the first and second jaw members defining a gap distance therebetween;
a sensor coupled to the housing, the sensor positioned proximal of the proximal end of the elongated shaft and longitudinally aligned with the proximal end of the elongated shaft along the longitudinal axis, the sensor configured to detect a position of the proximal end of the elongated shaft relative to the housing to determine the size of the gap distance, wherein, if the size of the gap distance is within a predetermined range, the first and second jaw members are configured to be electrically activated to treat tissue between the first and second jaw members,
wherein the sensor is configured to provide feedback that the size of the gap distal is within the predetermined range.

32. The surgical instrument according to claim 31, wherein the elongated shaft is translatable along the longitudinal axis to move the first and second jaw members relative to one another from a first configuration wherein the first and second jaw members are spaced relative to one another, to a second configuration wherein the first and second jaw members are closer to one another for approximating tissue.

33. The surgical instrument according to claim 30, wherein the proximal end of the elongated shaft is positioned proximal of the movable handle.

34. The surgical instrument according to claim 30, further including a drive assembly disposed within the housing and operatively associated with the moveable handle to longitudinally translate the elongated shaft.

35. The surgical instrument according to claim 30, wherein the drive assembly includes a tube having a distal end coupled to the proximal end of the elongated shaft and a proximal end longitudinally aligned with the sensor along the longitudinal axis.

36. The surgical instrument according to claim 35, wherein the drive assembly includes a biasing member configured to bias the elongated shaft distally.

37. The surgical instrument according to claim 31, wherein the feedback is audible, visual, or tactile.

38. The surgical instrument according to claim 31, further including a rotating assembly operably coupled with the elongated shaft to rotate the end effector with respect to the housing.

39. The surgical instrument according to claim 31, wherein the sensor is at least one of optical, magnetic, inductive, or mechanical.

40. The surgical instrument according to claim 31, wherein the sensor is fixedly coupled to the housing.
